(19) United States
(12) Patent Application Publication
(10) Pub. No.: US 2007/0113852 A1
(43) Pub. Date: May 24, 2007

(54) Snap-in deflector for respiratory mask

(76) Inventor: Christopher Martin, Springfield, OR (US)

Correspondence Address: CHRISTOPHER S. MARTIN, 445 S. 46TH, SPRINGFIELD, OR, 97478, US

(21) Appl. No.: 11/595,177

(22) Filed: Nov. 9, 2006

Related U.S. Application Data
(60) Provisional application No. 60/737,485, filed on Nov. 18, 2005.

(51) Int. Cl. A61M 16/00

(52) U.S. Cl. 128/204.24; 128/206.29

(57) ABSTRACT

The “Snap-in Deflector for Respiratory Mask” is a one piece plastic deflector held in place by its integral supports and snap ring (FIG. 2). It is designed to fit inside of a respiratory mask (FIG. 1). The deflector prevents a direct flow of air/gas into the mask user's airways without inhibiting or impeding the set flow or pressure of the device. The shape is consistent (FIG. 2); the size varies slightly and is determined by the size of mask it is to be installed in, e.g. small, medium, large. This deflector greatly reduces the irritation and discomfort of the interior of the mouth and nostrils associated with the use of respiratory masks.

This patent application claims priority to provisional patent application #60/737,485 filed Nov. 18, 2005.

### BACKGROUND OF THE INVENTION

In the art of the treatment of breathing disorders during sleep (sleep apnea), it is a common practice to implement the use of continuous positive air pressure (cpap) machines and related equipment. One such part of this equipment being used is a respiratory (cpap) mask. With this equipment outside air/gases are pumped into the respiratory mask at higher than atmospheric pressure to control sleep apnea. The respiratory masks, for this treatment, currently being manufactured are of such design that the flow of air/gas into them is directed into the mouth and nostrils of the wearer. This flow of air/gas into the airways causes irritation and discomfort to the wearer. This invention alleviates this condition.

### STATEMENT REGUARDING FEDERALLY SPONSORED RESEARCH.

Not applicable.

### REFERENCE TO SEQUENCE LISTING, OR A DISC APPENDIX

Not applicable

### BRIEF SUMMARY OF INVENTION

This invention is a plastic deflector installed inside respiratory masks. The deflector is installed without modification to the mask. The deflector is held in place by a single snap ring permanently attached to the deflector. The deflector is designed to prevent the flow of air/gas from directly entering the mouth and nostrils of the mask wearer. The design of the deflector is such that upon installation there is no added adjustment of the air/gas volume or pressure required. Installation of the deflector greatly reduces the discomfort and irritation of the airways of the mask user.

### BRIEF DESCRIPTION OF THE SEVERAL VIEWS OF THE DRAWING

FIG. 1 Side view showing placement location of deflector inside of facial mask.

FIG. 2 Side view of deflector only, showing shape of deflector and placement of deflector supports and deflector snap-ring.

FIG. 3 Top view of deflector showing orientation of supports and snap ring to deflector.

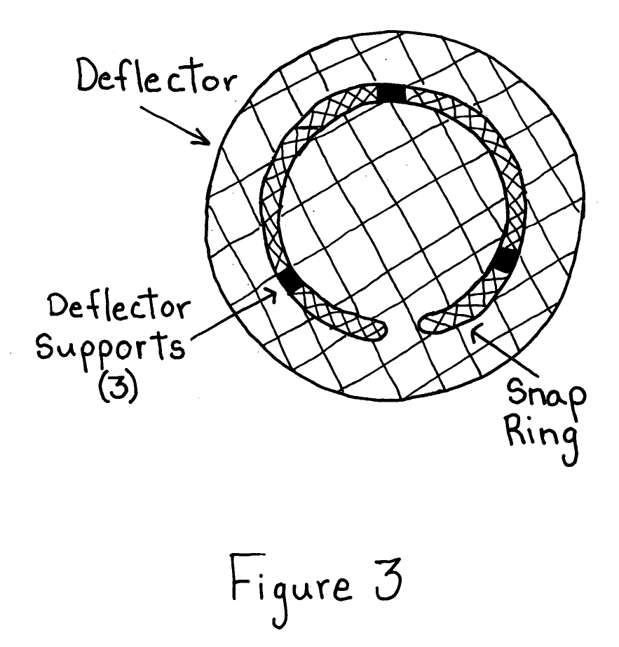

### DETAILED DESCRIPTION OF THE INVENTION

The deflector is round, approximately 1¾″ (one and three-quarters inches) in diameter, and convex. The deflector supports are approximately 7/16″ (seven-sixteenths inches) tall and placed at 120° (one hundred twenty degrees) from each other on the snap ring. The snap ring has an inside diameter of 13/16″ (thirteen-sixteenths inches) and an outside diameter of 1 3/16″ (one and three-sixteenths inches). The entire deflector assembly is one piece and made of semi-flexible injection molded composite plastic.

The deflector is secured inside the respiratory (cpap) mask by removing the existing lock ring from the valve elbow and replacing it with the deflector assembly.

The deflector size varies slightly depending on the size of the facial mask it is being used in, e.g. small, medium, large etc.

What is claimed is;

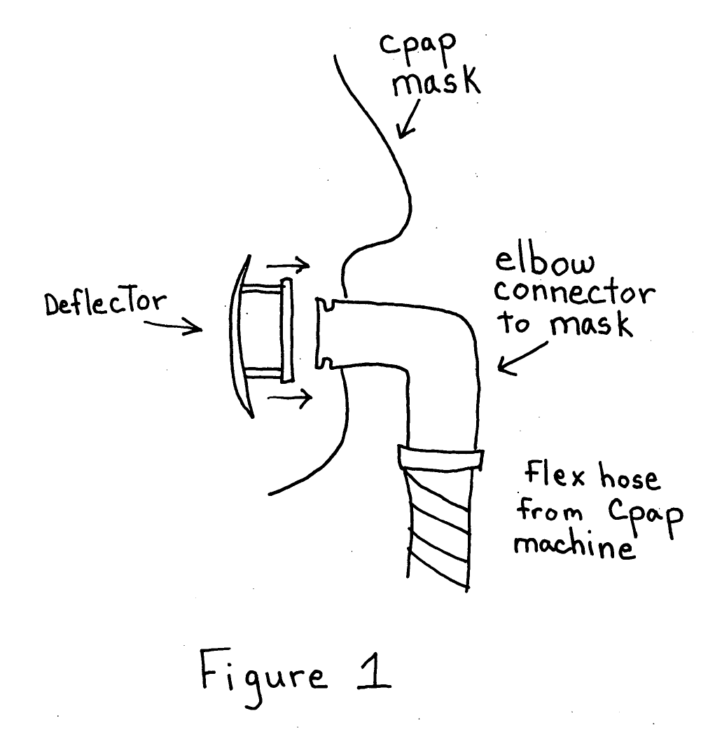

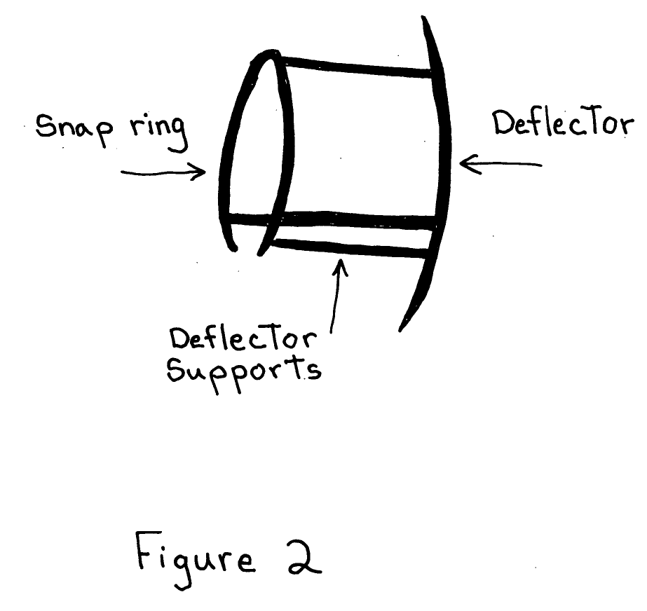

1. A deflection device of specific shape (FIG. 2) and mounting procedure (FIG. 1) for installation inside of a respiratory mask to ease, reduce or eliminate the flow of air/gas directly into the respiratory mask user's mouth and nostrils.

2. A deflector, as in claim 1, wherein installation and removal does not require modification of the respiratory mask.

3. A deflector, as in claim 1, does not inhibit nor impede the flow of air/gas and does not require adjustment to the air/gas volume or pressure, before, during, or after installation.

4. A deflector, as in claim 1, is easily, simply and quickly installed and removed by the mask user without specialized training.
